(19) United States
(12) Patent Application Publication
(10) Pub. No.: US 2023/0240612 A1
(43) Pub. Date: Aug. 3, 2023

(54) Self-Charging Dental Sensor System

(71) Applicant: Lydie Livolsi Tchouangang, Long Beach, CA (US)

(72) Inventor: Lydie Livolsi Tchouangang, Long Beach, CA (US)

(21) Appl. No.: 18/103,823

(22) Filed: Jan. 31, 2023

Related U.S. Application Data
(60) Provisional application No. 63/305,135, filed on Jan. 31, 2022.

(51) Int. Cl. A61B 5/00; A61C 5/70; A61B 5/291; A61B 5/01; A61B 5/145; A61B 5/1455

(52) U.S. Cl. CPC A61B 5/682; A61C 5/70; A61B 5/291; A61B 5/01; A61B 5/14539; A61B 5/14551; A61B 2560/0214; A61B 5/1112

(57) ABSTRACT

A dental sensor system has a dental appliance that is adapted, to be positioned within a mouth of a user. The dental appliance has at least one cavity within the dental appliance, a power generation system operably mounted on the dental appliance for generating power from movement of the user's mouth, and a battery mounted in the at least one cavity and operably connected to the power generation system. A sensor system is mounted in the at least one cavity and operably connected to the battery, and includes at least one sensor for sensing the data related to the user.

### CROSS-REFERENCE TO RELATED APPLICATIONS

This application for a utility patent claims the benefit of U.S. Provisional Application No. 63/305,135, filed Jan. 31, 2022.

### BACKGROUND OF THE INVENTION

### Field of the Invention

This invention relates generally to a sensor device, and more particularly to a dental sensor system that is adapted to be positioned in a user's mouth, and which includes a self-charging mechanism.

### Description of Related Art

Kimmel, U.S. Pat. No. 10,115,299, teaches an intraoral sensor appliance that is anchored to a tooth, or implanted in the mouth of a user. The appliance includes various forms of sensors, a memory storage device, a communications subsystem, an output device which creates stimulus directly or indirectly observable in the mouth environment; an input device, which can create signals according to activity in the mouth environment and can send them to the memory storage device and/or processor; and a processor coupled to the memory storage device. The appliance includes a battery and a power generator.

Logan, U.S. 2012/0172679, teaches a user monitoring system that includes a dental appliance configured to fit substantially inside a mouth of a user, a pulse oximeter for measuring user oxygen saturation level, a pressure sensor for measuring pressure exerted by the user and a processor for acquiring and processing oxygen saturation and pressure data of the user. The user monitoring system may communicate with an observer monitoring system. In some embodiments, the user monitoring system also measures one or more of ambient air temperature, light levels, respiration rate and environmental hazards.

Lee, U.S. 2020/0093436, teaches a tooth-attached wearable device that includes a body configured to be attached to a tooth, a sensor device, and is configured to sense biometric information of a patient. The device stores the biometric information sensed at the sensor device as data, and transmits the stored data. The device uses piezo electric elements for generating power for storage in the device battery.

Radmand, U.S. Pat. No. 11,191,663, teaches an oral appliance that includes various sensors for the treatment of obstructive sleep apnea. The appliance includes a mouthpiece configured for being received in an oral cavity of a user. The mouthpiece may include a pulse oximeter, a pressure sensor, an airflow sensor, an actigraphy sensor, a noise detector, and at least one stimulator for providing stimulation to a user's tongue in the event of decreased oxygen saturation levels, increased pressure applied to occlusal surfaces of the user's dentition, decreased actual airflow levels and/or increased noise levels. A microprocessor receives data from the oxygen sensor, pressure sensor, airflow sensor, actigraphy sensor and noise detector, and activates the at least one stimulator.

Saadat, U.S. 2013/0109932, teaches a system for determining a pulmonary function by mounting one or more sensors intra-orally; capturing intra-oral data; and determining the pulmonary function based on an analysis of the intra-oral data.

### SUMMARY OF THE INVENTION

The present invention teaches certain benefits in construction and use which give rise to the objectives described below.

The present invention provides a dental sensor system adapted to be positioned within a mouth of a user for sensing data related to a user. The dental sensor system includes a dental appliance for positioning within the mouth of the user; at least one cavity within the dental appliance; a power generation system operably mounted on the dental appliance for generating power from movement of the user's mouth; and a battery mounted in the at least one cavity and operably connected to the power generation system. A sensor system is mounted in the at least one cavity and operably connected to the battery, and includes at least one sensor for sensing the data related to the user.

A primary objective of the present invention is to provide a dental sensor system having advantages not taught by the prior art.

Another objective is to provide a dental sensor system that is able to self-charge so that it is wearable almost 24/7 and able to gather data continuously regarding brain activity, heart activity, blood chemistry, temperature, muscle activity, and other critical biometric data.

A further objective is to provide a dental sensor system that is able to transfer collected data to smart phones, tablets, and a remote computer to make the data collected accessible to the user as well as physicians and other health care providers.

A further objective is to provide a dental sensor system that is able to respond to recorded data with treatments, such as drug delivery, either from the dental sensor system, or an associated device.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention.

FIG. 1 is a perspective view of a dental sensor system according to one embodiment of the present invention.

FIG. 2 is a side elevational view of the dental sensor system of FIG. 1, once it has been installed in the mouth of a user.

FIG. 3 is a sectional view thereof taken along line 3-3 in FIG. 1.

FIG. 4 is an exploded sectional view of the dental sensor system of FIG. 3.

FIG. 5 is an exploded perspective view of a second embodiment of the dental sensor system, where the dental appliance is in the form of a denture.

FIG. 6 is a sectional view thereof taken along line 6-6 in FIG. 5.

FIG. 7 is an exploded side elevation view of a third embodiment of the dental sensor system, where the dental appliance is in the form of an implant.

FIG. 8 is a block diagram of one embodiment of an electronics package used in FIG. 7.

### DETAILED DESCRIPTION OF THE INVENTION

The above-described drawing figures illustrate the invention, a dental sensor system that is in the form of a dental appliance that is shaped and sized to be worn in the mouth of a user. As discussed in greater detail below, the dental sensor system includes one or more electronics packages that together include a power generation system, a rechargeable battery, a sensor system, and a communication system, all operably mounted within the dental appliance for collecting and transmitting data about a user wearing the dental sensor system. Since the dental sensor system is a self-charging system, it is able to operate continuously for unbroken data collection.

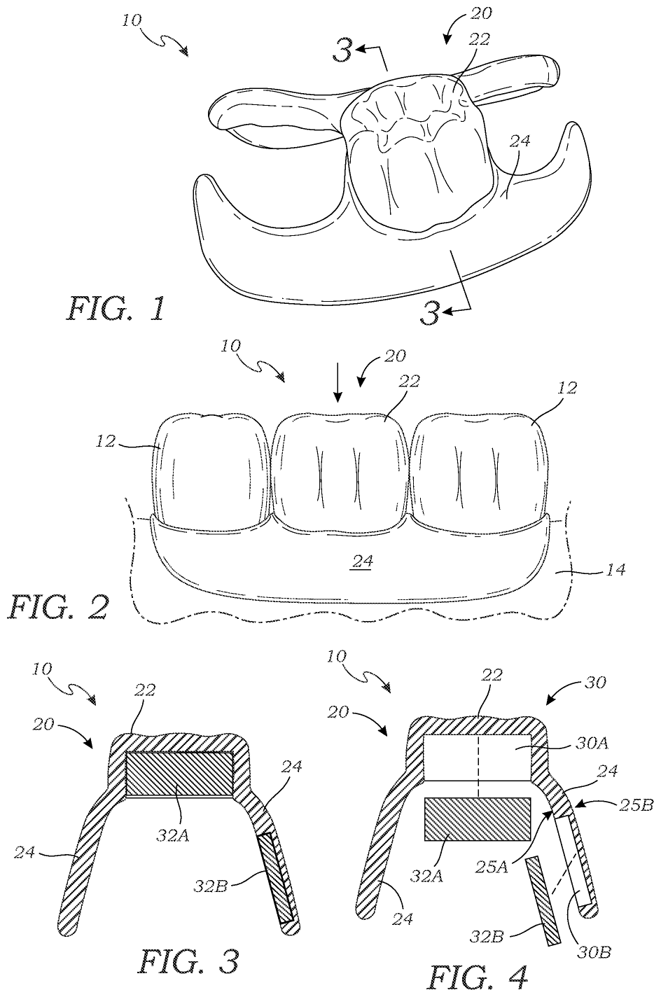

FIG. 1 is a perspective view of a dental sensor system 10 according to one embodiment of the present invention. As shown in FIG. 1, in this embodiment, the dental sensor system 10 has a dental appliance 20 that is in the form of a wearable device that fits over the user's gums, in this embodiment in the form of a flipper tooth (or other form of partial denture). The dental appliance 20 of this embodiment may include a simulated tooth 22 for replacing a missing tooth, and shaped sidewalls 24 that are shaped to abut the user's gums for holding the dental appliance 20 in place. The dental appliance 20 of this embodiment includes a molded plastic or acrylic (e.g., polypropylene or polyvinylchloride (PVC) material, or any other material known in the art for construction these types of dental appliances). The shaped sidewalls 24 extend downwardly from the simulated tooth 22 and fit over the gums of the user. The shaped sidewalls 24 may be custom molded to the user, using processes known in the art, or they may be mass produced to fit an average user. This device is typically custom molded using techniques well known in the art, and may include a flipper tooth as illustrated, or any other form of partial denture, and or any similar or equivalent appliance.

FIG. 2 is a side elevational view of the dental sensor system 10 of FIG. 1, once it has been installed in the mouth of a user. As shown in FIG. 2 the dental sensor system 10 may fit securely between the user's teeth 12 and around the use's gums 14 for holding the simulated tooth 22 in place. It may be shaped and installed to replace one or more teeth, and it may abut or be anchored to other teeth, gums. or other structures according to any of the methods known in the art.

FIG. 3 is a sectional view thereof taken along line 3-3 in FIG. 1. FIG. 4 is an exploded sectional view of the dental sensor system 10 of FIG. 3. As shown in FIGS. 3-4, the dental sensor system 10 may include a cavity or one or more cavities 30 in various locations of the dental sensor system 10 for receiving one or more electronics packages 32 that include the various electronics of the dental sensor system 10. For purposes of this application, the term “cavity” is broadly defined to include any form of recess, bore, compartment, or other structure that can each receive one of the electronics packages 32,

In the current embodiment, as best shown in FIG. 4, there are two cavities 30. A first cavity 30A is located under the tooth 22, and holds a first electronics package 32A. A second cavity 30B is located in an inner surface 25A of one of the shaped sidewalls 24 of the dental appliance 20, which receives a second electronics package 32B. In another embodiment, only one cavity and electronics package may be used, and the one or more may be located in any suitable location. They may, for example, alternatively be located on an outer surface 25B of one of the shaped sidewalls 24, or in any other location deemed suitable for the operation of the dental sensor system 10 and the comfort of the user. If there are more than one such electronics package, they may be electronically connected with a conductor (not shown), via a wireless connection, and/or any other form of operative connection known in the art.

As noted above, the electronics packages 32 collectively include or be operatively connected with a power generation system 40, a rechargeable battery 72, a communication system 94 such as a transceiver, and a sensor system 80 (all illustrated in FIG. 8), all operably mounted within the dental appliance 20 for collecting and transmitting data about the user wearing the dental sensor system 10. In this embodiment, the power generation system 40 is in the first electronics package 32A, and may include any form of power generation mechanism that is able to generate power from the operation of the user's mouth (e.g., chewing, swallowing. etc.). In this embodiment, the electronics package 32A includes a piezoelectric material that generates power from being deformed by the user chewing or otherwise using his or her mouth, generating power from the force applied by the user's teeth or gums against the dental appliance 20. The first electronics package 32A, in this embodiment, may also include the battery 72. The other operative components, shown in FIG. 8 and discussed in greater detail below, may be mounted in the second electronics package 32B. Obviously these components may be arranged in any manner deemed suitable by a product designer skilled in the art, and all such configurations should be considered within the scope of the present invention.

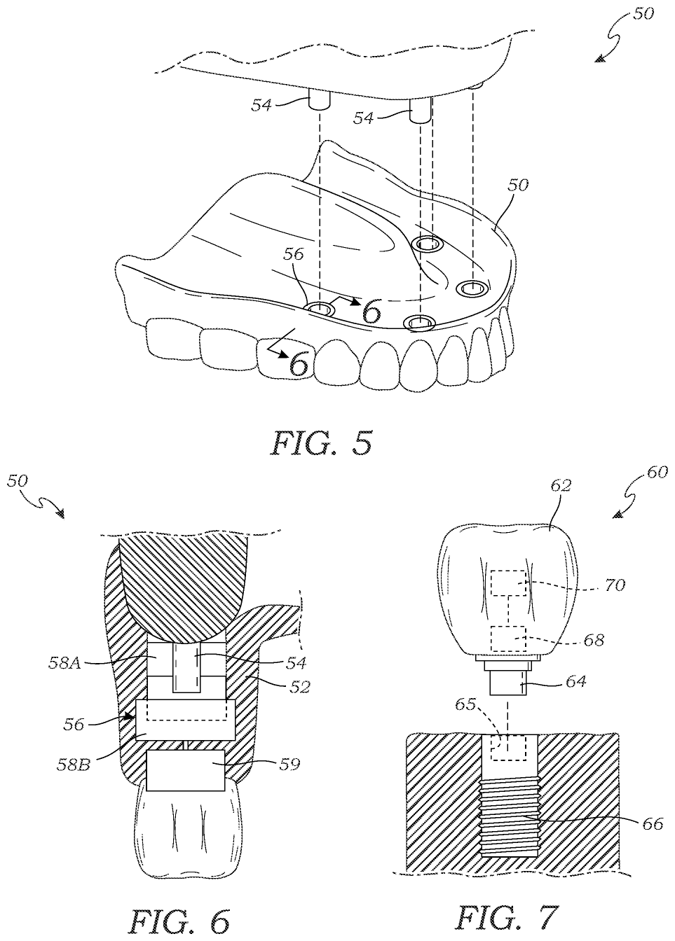

FIG. 5 is an exploded perspective view of a second embodiment of the dental sensor system 50, wherein the dental appliance 52 in the form of a denture. FIG. 6 is a sectional view thereof taken along line 6-6 in FIG. 5. As shown in FIGS. 5-6, in this embodiment, the denture 52 is adapted to be mounted onto posts 54 installed in the mouth (e.g., gums) of the user. In this embodiment, at least one of the posts 54 engages the power generation system 56. In this embodiment, the power generation system 56 includes a first components 58A and a second component 58B which move relative to each other and are configured to generate power when such movement occurs (e.g., via piezo electronic components, or some other form of energy generation mechanism known in the art). in this manner, when force is applied by the posts 54 against the power generation system 56, it generate power. Power generated is transferred via a conductor to the electronics package 59, which may include the battery and sensors etc. discussed in more detail elsewhere.

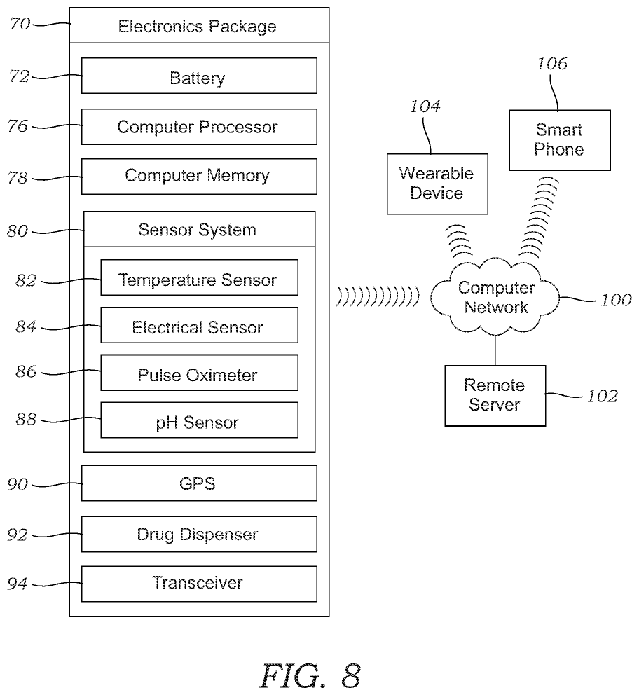

FIG. 7 is an exploded side elevation view of a third embodiment of the dental sensor system 60 where the dental appliance is in the form of a crown 62 mounted upon an abutment 64 which is mounted upon an implant body 66 (via insertion into bore 65) once the implant body 66 has been installed in the mouth of the user, using methods known in the art. In this embodiment, the crown 62 may include the power generation system 68 for generating power from movement of the user's mouth that apply forced upon the crown 62, as discussed above. This power generation system 68 supplies power to electronics package 69, which is mounted within the crown 62, or in other locations deemed suitable (e.g., abutment, implant, etc.).

FIG. 8 is a block diagram of one embodiment of the electronics package 70 of FIG. 7, although this obviously could be used, in different variations is needed, in any of the above-described embodiments. As shown in FIG. 8, the electronics package may include a battery 72 that is operably connected so that it may be charged by the power generation system, and for providing power to the rest of the system. The battery 72 may also be charged via an outside source, either wired or wireless charging (e.g., induction charging), or any other means for providing power known in the art.

As shown in FIG. 8, this may further include a computer processor 76 and a computer memory 78, which may be in the form of an electrical control circuit, or any other suitable circuitry that may be devised by one skilled in the art. While one embodiment of this device is illustrated, any computer circuitry known in the art for these purposes should be considered within the scope of the present invention.

For purposes of this application, the terms “computer,” “computer device,” “server,” and similar terms, refer to a device and/or system of devices that include at least one computer processor, and some form of computer memory having a capability to store data. The computer may comprise hardware, software, and firmware for receiving, storing, and/or processing data as described below. For example, a computer may comprise any of a wide range of digital electronic devices, including, but not limited to, a server, a desktop computer, a laptop, a smart phone, a tablet, or any form of electronic device capable of functioning as described herein.

The term “computer processor” as used herein refers to an electrical component that performs operations on an external data source, such as a computer memory, typically in the form of a microprocessor, although any equivalent structure may be used.

The term “computer memory” as used herein refers to any tangible, non-transitory storage that participates in providing instructions to a processor for execution. Such a medium may take many forms, including but not limited to, non-volatile media, volatile media, and any equivalent media known in the art. Non-volatile media includes, for example, ROM, magnetic media, and optical storage media. Volatile media includes, for example, DRAM, which typically serves as main memory. Common forms of computer memory include, for example, hard drives and other forms of magnetic media, optical media such as CD-ROM disks, as well as various forms of RAM, ROM, PROM, EPROM, FLASH-EPROM, solid state media such as memory cards, and any other form of memory chip or cartridge, or any other medium from which a computer can read. While several examples are provided above, these examples are not meant to be limiting, but illustrative of several common examples, and any similar or equivalent devices or system may be used that are known to those skilled in the art.

The electronics package 70 further include a sensor system 80 for gathering various forms of data based upon conditions which may be sensed within the user's mouth. For example, the sensor system 80 may include one or more sensors, such as a temperature sensor 82, an electrical sensor 84, a pulse oximeter 86, a pH sensor 88, and any other sensors known in the art. These are discussed in greater detail below. The sensors can include any forms of sensors known in the art for monitoring a user. They may be located in the dental sensor system 10, and them may also be located elsewhere (e.g., implanted, worn on clothing (or a smart watch, smart phone, etc.), or any other form of wearable sensor known in the art. Some options are discussed below, but this invention includes any other sensors known in the art.

For example, the sensors may allow:
1—Sensing the function of a user's brain, heart, blood, bones, sleep, muscles, body temperature, etc.
2—detect and monitor early symptoms of stroke, epilepsy, seizure, brain damage, tumors, encephalitis, mental retardation, sleep disorders, diabetes, Sjogren syndrome, tooth decay, degenerative diseases such as Alzheimer's disease and Parkinson's disease, alcoholism, schizophrenia, autism, different cancers, and also alerting the user or others if the user falls asleep while driving, or preventing the user from driving while drunk or otherwise impaired.
3—Measuring and delivering on-demand EEG on electronic devices like cellphones, watches etc. and in labs (Electroencephalogram), EKG (electro cardiogram), MRI, blood pressure, nerves, eyes, etc., from data collected and transmitted to a cloud.
4—If worn by astronauts in mission in the space, will provide more accurate, reliable and fast data about the astronaut's vitals and other desired measurements.
5—Potentially helps improving the performances of police, secret service agents, and other public servants.
6—Monitoring the operations of military personnel, including naval and submarine operations.
7—The device may be used on a dead person, because it still powered after a flat EEG, sensors will continue collecting and transmitting data to the cloud: this is the beginning of understanding what can happen after death.
8—The device may monitor body temperature.

The power generation system takes advantage of movements in the user's mouth to generate power. Biting and chewing can be used to generate power based upon the force of the bite. Furthermore, swallowing may be used in a similar manner. Each human being swallows between 1500 and 2000 times per day whether it's food, drinks or saliva. The mechanism of swallowing can be used to generate power. When we swallow, our mouth is closed, our teeth on both our upper and lower jaws come together in a strong contact, in the meantime, our tongue presses on our palate and we swallow.

Our brain cells that communicate via electrical impulses are active all the time even when we are asleep. The Electroencephalography (EEG) is a neurological test that uses an electronic device to measure and record electrical activity in the brain. Current EEG test is performed by small metal discs (electrodes) attached to our scalp lasts about 30 minutes. In fact, we get an EEG of our brain for about 30 minutes. EEG is often prescribed for stroke, epilepsy, seizure, brain damage, tumors, encephalitis, mental retardation, sleep disorders, alcoholism, autism, Alzheimer's and Parkinson's diseases, schizophrenia, autism, certain types of cancers etc. The stroke is the first scary death's cause after traffic accident. The cost of sleep tests is very expensive as they require hospitalization in order to get the test done during the night when the patient is asleep. In many traffic accidents' cause is either sleep while driving or DUI (driving Under Influence).

The electronics package 70 may further include a global positioning system (GPS) 90 for tracking the location of the user, which may be reported periodically for various monitoring systems.

The electronics package 70 may further include a drug dispenser 92 for dispensing drugs on a periodic basis, or in response to certain predetermined conditions. For example, if the sensors determine the user is having a health problem such as a heart attack, suitable drugs may be automatically be released to treat the problem, often before the user is even aware. A request for emergency assistance, or a report to a doctor or other professional or care provider, may also be transmitted.

A transceiver 94 may also be provided for electronic communications described herein. For purposes of this application, the term “transceiver” is broadly defined to include any form of transmitter or equivalent data transmitter, wireless preferred but not required, which may transfer data from the sensors via a network 100 to a remote computer 102, a wearable device 104 such as a smart watch. and/or a smartphone 106 or other suitable computer device (e.g., a desktop, laptop, tablet, etc.). The device 10 may communicate with any of these external devices for processing and storage of data, enabling the user and/or physician/caregiver to monitor data, and also respond to emergency conditions detected, either with prescriptions of drugs or other treatments, automatic dispensing of drugs or other treatments, alerting the user and emergency services if ambulatory services are required, interaction with the systems already in place in assisted living facilities, etc.

The title of the present application, and the claims presented, do not limit what may be claimed in the future, based upon and supported by the present application. Furthermore, any features shown in any of the drawings may be combined with any features from any other drawings to form an invention which may be claimed.

As used in this application, the words “a,” d “one” are defined to include one or more of the referenced item unless specifically stated otherwise. The terms “approximately” and “about” are defined to mean +/−10%, unless otherwise stated. Also, the terms “have,” “include,” “contain,” and similar terms are defined to mean “comprising” unless specifically stated otherwise. Furthermore, the terminology used in the specification provided above is hereby defined to include similar and/or equivalent terms, and/or alternative embodiments that would be considered obvious to one skilled in the art given the teachings of the present patent application. While the invention has been described with reference to at least one particular embodiment, it is to be clearly understood that the invention is not limited to these embodiments, but rather the scope of the invention is defined by claims made to the invention.

What is claimed is:

1. A dental sensor system adapted to be positioned within a mouth of a user for sensing data related to a user, the dental sensor system comprising:
a dental appliance for positioning within the mouth of the user;
at least one cavity within the dental appliance;
a power generation system operably mounted on the dental appliance for generating power from movement of the user's mouth;
a battery mounted in the at least one cavity and operably connected to the power generation system; and
a sensor system mounted in the at least one cavity and operably connected to the battery, the sensor system having at least one sensor for sensing the data related to the user.

2. The dental sensor system of claim 1, wherein the sensor system includes a temperature sensor.

3. The dental sensor system of claim 1, wherein the sensor system includes an electrical sensor.

4. The dental sensor system of claim 1, wherein the sensor system includes a pulse oximeter. The dental sensor system of claim 1, wherein the sensor system includes a pH sensor.

6. The dental sensor system of claim 1, wherein dental appliance is in the form of a flipper tooth.

7. The dental sensor system of claim 1, wherein dental appliance is in the form of a denture.

8. The dental sensor system of claim 1, wherein dental appliance is in the form of a crown.

9. The dental sensor system of claim 1, wherein power generation system includes a piezoelectric component.

10. The dental sensor system of claim 1, wherein power generation system is mounted in a simulated tooth and generates power based upon movements within the user's mouth.

11. The dental sensor system of claim 1, wherein power generation system is operatively connected with a rechargeable battery.

12. The dental sensor system of claim 1, further comprising a GPS for tracking a location of the user.

13. The dental sensor system of claim 1, further comprising a drug dispenser mounted in the dental appliance and operatively connected with an electronics package that receives power from the battery.

14. A dental sensor system adapted to be positioned within a mouth of a user for sensing data related to a user, the dental sensor system comprising:
a dental appliance in the form of a flipper tooth having a simulated tooth and shaped sidewalk for positioning the dental appliance within the mouth of the user;
at least one cavity within the dental appliance containing an electronics package;
a power generation system operably mounted on the dental appliance for generating power from movement of the user's mouth;
a battery mounted in the at, least one cavity and operably connected to the power generation system; and
a sensor system mounted in the at least one cavity as part of the electronics package and operably connected to the battery, the sensor system having at least one sensor for sensing the data related to the user.

15. The dental sensor system of claim 14, wherein the power generation system includes a piezoelectric component.

16. The dental sensor system of claim 14, wherein power generation system is mounted in a simulated tooth and generates power based upon movements within the user's mouth.

17. The dental sensor system of claim 14, further comprising a GPS for tracking a location of the user.

18. The dental sensor system of claim 14, further comprising a drug dispenser mounted in the dental appliance and operatively connected with the electronics package that receives power from the battery.
